(19) United States
(12) Patent Application Publication
(10) Pub. No.: US 2009/0018387 A1
(43) Pub. Date: Jan. 15, 2009

(54) Repair of Vaginal Prolapse

(76) Inventor: Dionysios K. Veronikis, Town of Country, MO (US)

Correspondence Address: AMS RESEARCH CORPORATION, 10700 BREN ROAD WEST, MINNETONKA, MN, 55343, US

(21) Appl. No.: 12/170,856

(22) Filed: Jul. 10, 2008

Related U.S. Application Data
(60) Provisional application No. 60/948,847, filed on Jul. 10, 2007.

(51) Int. Cl. A61F 2/00

(52) U.S. Cl. 600/37

(57) ABSTRACT

Described are methods and devices useful for treating pelvic prolapse, such as vaginal prolapse, the methods involving a tissue path that extends through pelvic floor (muscle) tissue.

The present non-provisional patent Application claims priority under 35 USC §19(e) from U.S. Provisional Patent Applications having Ser. No. 60/948,847, filed on Jul. 10, 2007 by Veronikis and titled REPAIR OF VAGINAL PROLAPSE, wherein the entirety of said provisional patent application is incorporated herein by reference.

### FIELD OF THE INVENTION

Described herein are features of surgical articles, surgical methods, and surgical tools, for use in treating vaginal prolapse.

### BACKGROUND

Vaginal prolapse includes more specific conditions referred to as vault prolapse (apical), enterocele, cystocele (anterior), rectocele (posterior), and combinations of these. Various techniques have been designed to correct or ameliorate vaginal vault prolapse and its symptoms, with varying degrees of success. Nonsurgical treatments involve measures to improve the factors associated with prolapse, including treating chronic cough, obesity, and constipation. Other nonsurgical treatments may include pelvic muscle exercises or supplementation with estrogen.

A variety of surgical procedures have also been attempted for the treatment of pelvic vaginal prolapse. See for example U.S. patent application Ser. No. 10/834,943, entitled “Method and Apparatus for Treating Pelvic Organ Prolapse,” filed Apr. 30, 2004; U.S. patent application Ser. No. 11/398,368, entitled “Articles, Devices, and Methods for Pelvic Surgery,” filed Apr. 5, 2006; and U.S. patent application Ser. No. 10/431,719, entitled “Implantable Article and Method,” filed May 8, 2003. Such patent applications describe articles and methods for pelvic organ prolapse by use of a support member for supporting specific tissue.

### SUMMARY OF THE INVENTION

The invention relates to a new and improved method for treating vaginal prolapse, especially posterior vaginal prolapse such as vaginal vault prolapse and rectocele.

The present method involves the use of an implant to support tissue of the vagina. The implant contacts vaginal tissue, e.g., posterior vaginal tissue, extends from the vaginal tissue to a location within the pelvic region, and connects to tissue of the pelvic region to support the contacted vaginal tissue.

According to exemplary methods of the invention, the implant connects to tissue of the pelvic region by being placed in a tissue path that tunnels through muscle tissue of the pelvic floor. The tissue path enters the muscle tissue from the interior side of the pelvic floor, passes through (i.e., “tunnels” through) a length of the muscle tissue, then exits the muscle in a direction to re-enter the pelvic floor. Certain previous tissue paths involved in treating vaginal prolapse may pass through muscle tissue, but they are not known to enter the muscle tissue from the interior side of the pelvic muscle, tunnel through the tissue, and re-enter the interior of the pelvic region; instead, previous tissue path pass through entering on one side of a muscle tissue and exiting on the other, at the same general placement on the muscle.

An exemplary tissue path according to the invention can extend through tissue of levator or coccygeus muscle (or a combination of these) between an inferior location of muscle that is below (i.e., inferior to) the sacrospinous ligament and a superior location of muscle that is at or superior to a level of the sacrospinous ligament. The superior location can optionally be as far superior as the sacrotuberous ligament or fascia or perineum of the sacrum.

As used herein, the terms “superior” and “inferior” are used to refer to their common anatomical meanings. “Superior” means above, toward the head, or “cranial”; inferior means below or lower, away from the head or “caudal.”

The method can be performed transvaginally and without the need for any external incision. Advantages can include the elimination of external skin punctures and incisions; the variable depth of repair available; use of a pulley elevating mechanism; either unilateral or bilateral elevation; expansive and preferably complete surgical visualization with no blind spots; minimal instrumentation; and an overall more simple technique compared to other methods of supporting vaginal tissue.

The following U.S. patents and publications are herein incorporated by reference: U.S. Pat. No. 6,911,003, (U.S. Ser. No. 10/377,101) to Anderson et al., “Transobturator Surgical Articles and Methods,” filed Mar. 3, 2003; U.S. patent publication number 2004/0039453 (U.S. Ser. No. 10/423,662) to Anderson et al., “Pelvic Health Implants and Methods,” filed Apr. 25, 2003; U.S. patent publication number 2005/0245787 (U.S. Ser. No. 10/834,943) to Cox et al., “Method and Apparatus for Treating Pelvic Organ Prolapse,” filed Apr. 30, 2004; and U.S. Pat. No. 7,351,197, (U.S. Ser. No. 10/840,646) to Montpetit et al., “Method and Apparatus for Cystocele Repair,” filed May 7, 2004.

An aspect of the invention relates to a method for supporting vaginal tissue. The method includes: providing an implant, creating a vaginal incision at a posterior vaginal wall; accessing muscle tissue of a pelvic floor; creating a tissue path through muscle tissue of the pelvic floor, the tissue path extending through muscle tissue between a location inferior to a sacrospinous ligament and a location at a level of the sacrospinous ligament; placing the implant through the tissue path; placing the implant in contact with vaginal tissue in a position to support the vaginal tissue and adjusting the implant to support the vaginal tissue.

In another aspect, the invention relates to a method for supporting vaginal tissue. The method includes: providing an implant; creating a vaginal incision at a posterior vaginal wall; and accessing muscle tissue of a pelvic floor. A tissue path is created through muscle tissue of the pelvic floor by entering muscle tissue of the pelvic floor at a location inferior to the sacrospinous ligament, extending the tissue path in a superior direction by tunneling toward the sacrospinous ligament, and exiting the muscle tissue. The implant is placed in the tissue path, and the implant is placed in contact with vaginal tissue in a position to support the vaginal tissue. The implant is adjusted to support the vaginal tissue.

### BRIEF DESCRIPTION OF THE DRAWINGS

FIG. 1 illustrates an example of an implant assembly.

FIG. 2 illustrates certain anatomy of the pelvic region.

### DETAILED DESCRIPTION

The invention involves placement of a pelvic implant to support tissue of the vagina, e.g., posterior vaginal tissue, to treat conditions of vaginal prolapse, especially posterior vaginal prolapse such as vaginal vault prolapse, rectocele, and combinations of these.

In general, an implant can include a tissue support portion that can be used to contact tissue of the vagina, to support the tissue. During use, the tissue support portion can be placed in contact with and attached to vaginal tissue such as with a suture or other securing mechanism. An implant can additionally include one or more “extension” portions (also sometimes referred to as an “end portion,” “arm,” or “leg” of an implant). The extension portion is generally elongate and has a distal and a proximal end. The proximal end attaches to the tissue support portion and the distal end can be extended from the tissue support portion to contact and attach to tissue in a patient's pelvic region, to thereby support the tissue support portion and the vaginal tissue to which the tissue support portion is attached.

An implant may include sections that are synthetic or of biological material (e.g., porcine, cadaveric, etc.). End portions may be, e.g., a synthetic mesh such as polypropylene. The tissue support portion may be synthetic (e.g., a polypropylene mesh) or biologic. Examples of implant products that are sold commercially include a number sold by American Medical Systems, Inc., of Minnetonka Minn., under the trade names Apogee® and Perigee® for use in treating pelvic prolapse (including vaginal vault prolapse, cystocele, enterocele, etc.).

An example of a particular type of pelvic implant is the type that includes supportive portions including or consisting of a central support portion and two elongate extension portions extending from the central support portion. The term “supportive portions” refers to portions of an implant that function to support tissue after the implant has been implanted, and specifically includes extension portions and tissue support portions, and does not include optional or appurtenant features of an implant such as a sheath or dilator.

An extension portion can have dimensions to extend from the tissue support portion when attached to tissue of the vagina, to a tissue entry as described, extending through a length of a tissue path as describe, exiting a second tissue entry, with additional length for material to extend beyond the second tissue entry for adjusting the location or tension of the implant. The length of an extension portion can be measured as from a location where an end portion meets a tissue support portion, to an opposite distal end of the extension portion. Exemplary lengths of an extension portion may be from 10 to 20 centimeters, e.g., from 13 to 17 centimeters.

A tissue support portion can be of equal width as an extension portion, or of a greater width. If the tissue support portion is the same width as extension portions, the implant material is in the form of a single elongate mesh strip. Exemplary lengths of such a mesh strip can be from 21 to 37 centimeters.

A width of an extension portion (and optionally a tissue support portion) can be a width useful for implanting the implant and for providing desired strength and fixation properties during and after implantation and optional adjusting and tensioning of the sling. Typical widths of end portions can be in the range from 7 to 10 centimeters. Extension portions can typically have a uniform or substantially uniform width along the length.

An implant can optionally include a sheath that covers an extension portion. A sheath can cover a portion or entire length of an extension portion of an implant to facilitate installation by allowing the sheath (covering an extension portion, such as a mesh extension portion) to be inserted into and through a tissue path with reduced friction relative to the friction that would occur by directly inserting the implant material. Further, the sheath allows a surgeon to apply tension or pressure on the sheath, optionally to indirectly pressure or tension the extension portion or tissue support portion. This allows the surgeon to adjust the implant to achieve desired position and tension. After adjusting, the sheath can be removed to allow the implant material (e.g., mesh) to frictionally engage the tissue. A sheath can be of any flexible material such as plastic or paper, and preferably can be a transparent plastic tube or envelope that covers a length of an extension portion and can be removed at a desired time by a surgeon.

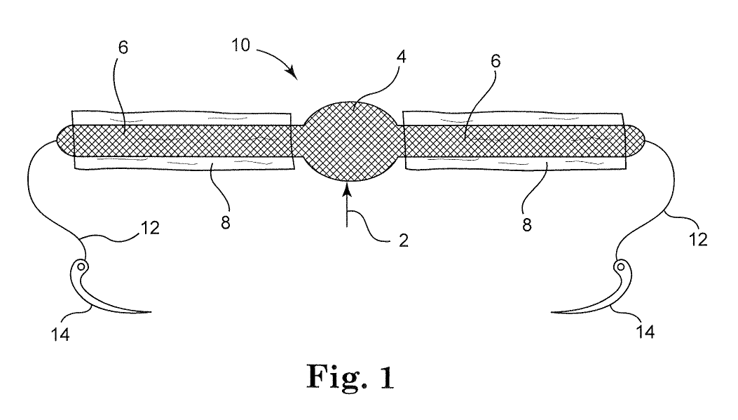

FIG. 1 illustrates a single example of an implant assembly that can be useful according to the described method. Variations of this and other implants will also be useful. Referring to FIG. 1, implant assembly 10 includes implant 2, which consists of tissue support portion 4 and extension portions 6. Tissue support portion 4 is illustrated to be of a greater width than the extension portions, but may alternately be of the same width. Transparent plastic sheaths 8 cover each extension portion 6. Sutures 12 extend from distal ends of extension portions 6, and each is connected to needle 14.

Pelvic floor musculature is the muscle tissue of the pelvic floor that forms a lower (inferior) support for the pelvic region. Particular muscles include levator muscle (sometimes referred to as “levator ani”) and muscles that make up the levator muscle (puborectalis, pubococcygeus, and iliococcygeus), and the coccygeus (also known as ischio-coccygeus) which is generally posterior to the levator ani.

The pelvic floor includes a posterior region that is located posterior and posterior-to-lateral, relative to the vaginal apex, on a right side of a patient and on a left side of a patient. Tissue of this posterior region includes medial tissues at and surrounding the sacrum and coccyx bones, rectum, and pararectal space, and tissue and features that extend in a lateral direction to and including the ischial spine. Tissues of the pelvic floor posterior and lateral to the vaginal apex include the coccygeus muscle, levator ani (e.g., puborectalis and pubococcygeus muscles) sacrospinous ligament, sacrotuberous ligament, and fascia and periostium of the sacrum.

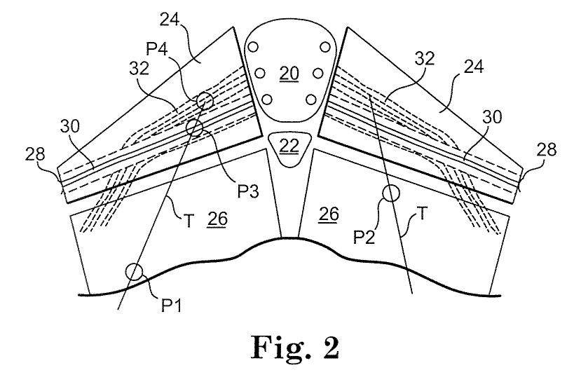

FIG. 2 illustrates certain anatomy that is relevant to the described methods. FIG. 2 shows sacrum 20, coccyx 22, and left and right ischial spines 28. (FIG. 2 is drawn to show the “interior” side of the pelvic region, which is the side of the pelvic region that contains organs and tissues including the vagina and rectum.) Coccygeus muscles 24 each extend from medial attachments at the sacrum to lateral attachments at the ischial spine. Inferior to coccygeus muscles are levator muscles 26 (including pubococcygeus muscle). Posterior to coccygeus muscles 24 are sacrospinous ligaments 30 and sacrotuberous ligaments 32 (each shown in dashed lines), each attached medially at sacrum 20 and extending in a lateral direction to ischial spines 28 and ischial tuberosities (not shown), respectively. Not shown, but relevant, are tissues of the rectum, and periosteum and fascia of the sacrum.

According to methods described herein, an implant can be implanted to place a tissue support portion in contact with vaginal tissue, with an extension portion being located internally to support the tissue support portion, which in turn supports the vaginal tissue. The extension portion can be located in a tissue path created in muscle of the posterior pelvic region, such as muscle of the coccygeus muscle or pubococcygeus muscle. The extension portion enters the tissue path from the internal side of the pelvic region, tunnels through the tissue path along a length of the muscle, and then exits the muscle in a direction back toward the interior of the pelvic region (i.e., re-enters the interior pelvic region). Preferably, the tissue path can extend through muscle of the pelvic floor from a location below the sacrospinous ligament, in a direction toward the sacrospinous ligament, to a location that is in line with the sacrospinous ligament or superior to the sacrospinous ligament, such as in line with the sacrotuberous ligament, or extending to fascia or periosteum of the sacrum. An implant can be supported by one or by two extension portions placed in the pelvic floor tissue, either on a patient's right side, a patient's left side, or on both a right and a left side.

Generally, an exemplary method can include a step of creating a vaginal incision at a posterior vaginal wall, then dissecting or moving tissue such as the rectum, to access the pararectal space and muscle tissue of a pelvic floor. A tissue path is created in muscle of the pelvic floor. An a portion of the implant is placed in the tissue path by entering the tissue path on the interior side of the muscle (entering from the interior of the pelvic region), tunneling through a length of the muscle, then exiting the muscle on the same side of the muscle that the implant entered (to re-enter the interior of the pelvic region). The tissue support portion is contacted with vaginal tissue. The implant is positioned and adjusted and tensioned if necessary to support the vaginal tissue.

A tissue path can be created by any method. A useful method is by the use of a needle with an attached suture, the suture also being attached to the implant. The needle can enter muscle tissue of the pelvic floor (from the interior of the pelvic region), be pulled through the muscle while pulling the suture, and the implant is pulled into place in the tissue path. The needle exits the tissue path at a desired location, returning to the interior of the pelvic region. The tissue path extends through muscle tissue of the pelvic floor, between two points of tissue entry. A tissue “entry” means a location where an implant extends from the pelvic region interior into muscle tissue. A tissue path is referred to as having two “entries” even though as a practical matter an implant will be inserted into (i.e., enter) one of the “entries,” pass through the tissue path, then exit through the other “entry.”

Preferred tissue paths can begin at a position that is below the sacrospinous ligament, e.g., by inserting a needle into tissue of the coccygeus or pubococcygeus muscle from the interior of the pelvic region. FIG. 2 illustrates exemplary such tissue entries, identified as P1 and P2. These tissue entries are below (inferior to) sacrospinous ligaments 30. From tissue entries P1 and P2, a tissue path can be created through tissue of the levator muscle (26) and coccygeus, in a trajectory that extends toward and optionally past sacrospinous ligaments 30, e.g., in a trajectory toward the sacrum. As illustrated at FIG. 2, trajectories T are lines that extend through muscle tissue in a direction superior to tissue entry P1 or P2. The tissue path may end at a location that is level with a sacrospinous ligament, such as to tissue entry P3, or that is superior to a sacrospinous ligament, e.g., level with a sacrotuberous ligament, such as to tissue entry P4. Alternately, the tissue path may exit the muscle tissue at any other point along the trajectory, such as at fascia of the sacrum, or periosteum of the sacrum.

The depth at which the tissue path passes through the muscle can be as desired, with an exemplary depth being from 3 to 7 millimeters below the muscle tissue surface. The total length of the tissue path, through pelvic floor muscle, can be sufficient to secure an extension portion of an implant.

In more detail, an example of steps of an embodiment of a method as described can include the following.

Once the posterior vaginal wall has been incised, the rectovaginal space is developed, facilitating entry into the pararectal space with further development by mobilizing the bladder superiorly and the rectum medially which further exposes the pelvic floor with the pubococcygeus, coccygeus, sacrospinous ligament, sacrotuberous ligament are exposed and visualized, as well as the fascia/periosteum of the sacrum.

After accounting for individual patient anatomy and structures that may be scarred from obstetrical deliveries an entry penetration can be made (e.g., using a surgical needle, attached to a suture, attached to an implant) directly into the pelvic floor musculature and carried/tunneled over a desired length through the muscle tissue toward the sacrospinous ligament and if desired further to the sacrotuberous or to the fascia or periosteum of the sacrum. The depth at which the tissue path is placed can depend on surgeon choice based on tissue quality and the desire to maintain direct visual contact with the desired trajectory of the tissue path between entry points. A depth of from 3 to 7 millimeters below the surface of the muscle tissue can be adequate.

At the exit penetration, the suture is recovered by any useful method. Once the suture is recovered it will now traverse from the outside of the patient's body transvaginally into the levator/pelvic floor at the entry site, travel through the levators/pelvic floor to exit at the sacrospinous or sacrotuberous or the fascia/periosteum of the sacrum or at any point along the trajectory chosen by the surgeon. The mesh (e.g., measuring 7 mm to 10 mm in diameter and 21 cm to 37 cm), encased in plastic to prevent premature tissue deployment, is secured to the suture and pulled halfway through the pelvic floor. (A mesh strip implant can be used, or other versions of the implant, such as embodiments that incorporate bigger pieces of mesh for repairing not only the apex (level 1) but also rectocele condition (level 2)).

The implant is in position and can be secured to the vaginal apex (or other vaginal tissue) for repair of the vaginal vault prolapse. The timing and sequence of this step can depend on surgeon choice as well as whether other defects are being repaired during the same surgical procedure. The end of the mesh that has not yet entered the pelvic floor is secured/sewn to the vaginal apex, and portion of the implant (e.g., mesh extension portion) that has been pulled through the pelvic floor, protected by the plastic sheath, is pulled to adjust the placement and tension of the implant. At the desired time and traction force, the plastic sheath is deployed (removed) allowing the mesh to self secure into the pelvic floor and support/stabilize and elevate the vaginal apex. The extra mesh extending from tissue exiting the pelvic floor muscle, is excised. The pelvic floor functions as a pulley mechanism around which the mesh travels and the traction on the mesh/sheath complex elevates the vaginal apex. The procedure may be performed on the left, right, or bilaterally.

This invention can also relate to kits for prolapse repair containing mesh, as described, a plastic sheath that covers portions of the mesh, needle, introducers (e.g., needles) or sutures.

1. A method for supporting vaginal tissue, the method comprising
providing an implant,
creating a vaginal incision at a posterior vaginal wall,
accessing muscle tissue of a pelvic floor,
creating a tissue path through muscle tissue of the pelvic floor, the tissue path extending through muscle tissue between a location inferior to a sacrospinous ligament, to a location at a level of the sacrospinous ligament,
placing the implant through the tissue path,
placing the implant in contact with vaginal tissue in a position to support the vaginal tissue and adjusting the implant to support the vaginal tissue.

2. A method for supporting vaginal tissue, the method comprising
providing an implant,
creating a vaginal incision at a posterior vaginal wall,
accessing muscle tissue of a pelvic floor,
creating a tissue path through muscle tissue of the pelvic floor by
entering muscle tissue of the pelvic floor, from the pelvic region interior, at a location inferior to the sacrospinous ligament,
extending the tissue path in a superior direction by tunneling toward the sacrospinous ligament,
exiting the muscle tissue in a direction of the pelvic region interior,
placing an implant in the tissue path,
placing the implant in contact with vaginal tissue in a position to support the vaginal tissue and adjusting the implant to support the vaginal tissue.

3. A method according to claim 2 wherein the tissue path extends through coccygeus muscle.

4. A method according to any of claims 2 wherein the tissue path extends through levator muscle.

5. A method according to any of claims 2 wherein the tissue path extends between a tissue entry inferior to the sacrospinous ligament and a tissue entry at a level of the sacrospinous ligament.

6. A method according to any of claims 2 wherein the tissue path extends between a tissue entry inferior to the sacrospinous ligament and a tissue entry at a level of the sacrotuberous ligament.

7. A method according to any of claims 2 wherein the tissue path extends between a tissue entry inferior to the sacrospinous ligament and a tissue entry at a level of the sacrotuberous ligament.

8. A method according to any of claims 2 wherein the tissue path extends between an inferior tissue entry that is inferior to the sacrospinous ligament and a superior tissue entry at a point along a line between the inferior tissue entry and sacrum.

9. A method according to any of claims 2 wherein the implant comprises
a tissue support portion,
a mesh extension portion connected at a proximal end to the tissue support portion,
a suture connected to a distal end of the mesh extension portion,
a plastic sheath covering at least a portion of the mesh extension portion.

10. The method of claim 9 comprising placing the extension portion through the tissue path while the plastic sheath covers at least a portion of the mesh extension portion, adjusting the implant to support the vaginal tissue, and removing the mesh after adjusting.

11. A method according to any of claims 2, wherein the method is for treatment of vaginal vault prolapse.

12. A method according to any of claims 2, wherein the method is for treatment of rectocele.
